(19) United States
(12) Patent Application Publication
(10) Pub. No.: US 2025/0250092 A1
(43) Pub. Date: Aug. 7, 2025

(54) A MULTI-COMPARTMENT MIXING CONTAINER SYSTEM

(71) Applicant: Alexandre Charbel EL KHOURY, Al Fanar (LB)

(72) Inventor: Alexandre Charbel EL KHOURY, Al Fanar (LB)

(21) Appl. No.: 18/695,066

(22) PCT Filed: Oct. 4, 2023
(86) PCT No.: PCT/IB2023/059944

§ 371 Date: Mar. 25, 2024

(30) Foreign Application Priority Data
Oct. 4, 2023 (WO) PCT/IB2023/059944

(51) Int. Cl. B65D 81/32; A61L 2/10; B65D 81/38

(52) U.S. Cl. CPC B65D 81/32; A61L 2/10; B65D 81/38; B65D 2203/12

(57) ABSTRACT

A multi-compartment mixing container system configured to blend or mix contents. The container system comprising n container (101), an inner container (104), inner screw tubes (102a-102b), a sip-and-seal lid (103b), a disinfecting module (107), a stirring mechanism, a phase change material (106) placed within the toroidal detachable ring (111) and fills the gap between the inner screw tubes and the container (101), a processing module (108), a temperature sensitive paint (1029), a user interface module (1014a). The temperature sensitive paint (1029) on the surface of the container (101) is used to indicate the temperature of the container system (100). The container (101) includes built-in grooves (102c). The inner screw tubes (102a-102b) are designed to fit into these grooves (102c) within the container (101) and are configured to store one or more contents separately.

### FIELD OF THE INVENTION

The present invention relates to a multi-compartment mixing container system with an inner removable screw tube, which enables the separation and subsequent blending of two or more materials, such as solids, powders, and liquids, in a single, convenient vessel. The container also has a variety of advanced features, such as thermal regulation, disinfection capabilities, electronic controls, and connectivity. These features make the container a versatile and efficient solution for mixing a wide range of substances.

### BACKGROUND OF THE INVENTION

Conventional beverage containers, such as water bottles, tumblers, and thermoses, are typically designed to hold a single type of beverage at a single temperature. While these containers fulfil their primary purpose of storing and transporting beverages, they are often not well-suited to meet the diverse and ever-changing needs of today's consumers.

These conventional containers lack the capability to store and mix different materials effectively. Consumers often desire personalized drink combinations that incorporate solids, powders, and liquids, yet no existing product seamlessly enables such customization. This invention addresses the need for a dual-compartment mixing cup that empowers users to create their desired blends at the moment of consumption. It uniquely combines storage and mixing functionalities, allowing users to turn different materials into a single mix just before consuming.

Therefore, there is a need in the art for a container that goes beyond basic storage, offering advanced features such as temperature regulation, multiple compartments for different ingredients, and electronic components for monitoring and control. This invention fills this gap, bringing together innovative design and technological sophistication to elevate the user experience in storing and mixing consumables. It offers the freedom of customization along with the assurance of quality and safety, setting a new standard in the field of portable beverage containers.

The present application is described hereinafter by various embodiments. This application may, however, be embodied in many different forms and should not be construed as limited to the embodiment set forth herein.

### SUMMARY OF THE INVENTION

According to a first aspect of the present application, there is provided a multi-compartment mixing container system for mixing multiple contents at a predefined temperature range. The container system comprises, but not limited to, a container, a sip-and-seal lid, a toroidal removable ring, one or more inner screw tubes, a stirring mechanism, a phase change material placed, a temperature sensitive paint, a disinfecting module, a user interface module and a processing module. The container includes built-in one or more grooves inside. The sip-and-seal lid is configured to cove on the top of the container. The toroidal removable ring situated within the sip-and-seal lid. The one or more inner screw tubes adapted to fit into the one or more grooves within the container. The each of the inner removable screw tubes configured to separately store one or more contents. The phase change material disposed within the toroidal removable ring and in the space between the inner removable screw tubes and the container. The temperature sensitive paint on the surface of the container to indicate the temperature of the container. The disinfectant module with a UV light unit and/or an ultrasonic unit, placed proximal to the sip-and-seal lid on the bottom side of the container, to disinfect the inner surfaces of the container. The user interface module disposed the container or the sip-and-seal lid, includes a touch sensitive display unit. The touch sensitive display unit with one or more touch sensitive areas or input unit. The stirring mechanism to facilitate mixing of the one or more contents stored in the one or more inner screw tubes by twisting or turning along the one or more grooves up and down in the container; processing module connected to a battery module; wherein the processing module operatively connected to the user interface module, and the disinfectant module. The processing module is configured to: receive input from the user interface module; process the input; and switch the disinfectant module and the touch sensitive display unit.

In accordance with an embodiment of the present invention, the touch sensitive display unit is configured to show a clock, a timer, warnings, clock time, stopwatch, a timer configured to notify users of optimal mixing times and displays nutritional values for combined ingredients.

In accordance with an embodiment of the present invention, the user interface module includes a sound unit, includes one or more input buttons, and, a communication module.

In accordance with an embodiment of the present invention, the one or more input buttons are selected from touch sensitive buttons, proximity switches, SPOT, DPDT or push buttons.

In accordance with an embodiment of the present invention, the container is doubly insulated and includes a translucent section and level indicator markings for ingredient level monitoring.

In accordance with an embodiment of the present invention, the temperature sensitive paint on the surface of the container is configured to display the degree of hotness and coldness or temperature by varying between lighter and darker shades of colour to indicate temperature.

In accordance with an embodiment of the present invention, the container and the one or more inner screw tubes are foldable for convenient storage and transport.

In accordance with an embodiment of the present invention, the phase change material is selected from salt hydrate mixtures, eutectic mixtures, paraffin wax, potassium nitrate, fatty acids, organic based compounds or combination thereof.

### BRIEF DESCRIPTION OF THE DRAWINGS

The accompanying Figures (Figs.) illustrate embodiments and serve to explain principles of the disclosed embodiments. It is to be understood, however, that these Figures are presented for purposes of illustration only, and not for defining limits of relevant applications. It is to be noted, however, that the appended drawings illustrate only typical embodiments of this application and are therefore not to be considered limiting of its scope, for the application may admit to other equally effective embodiments.

These and other features, benefits, and advantages of the present application will become apparent by reference to the following text Figure, with like reference numbers referring to like structures across the views, wherein:

FIG. 1A illustrates a multi-compartment mixing container system for storing and mixing multiple contents at a predefined temperature range, in accordance with an embodiment of the present invention;

FIG. 1B illustrates one or more inner removable screw tubes 102a-102b of multi-compartment mixing container system, in accordance with an embodiment of the present invention;

FIG. 1C illustrates an inner container of the multi-compartment mixing container system, in accordance with an embodiment of the present invention;

FIG. 2A illustrates top view of sip-and-seal lid of the multi-compartment mixing container system, in accordance with an embodiment of the present invention;

FIG. 2B illustrates bottom view of sip-and-seal lid of the multi-compartment mixing container system, in accordance with an embodiment of the present invention; and

FIG. 3 illustrates functional of multi-compartment mixing container system, in accordance with an embodiment of the present invention.

### DETAILED DESCRIPTION OF THE DRAWINGS

While the present invention is described herein by way of example using embodiments and illustrative drawings, those skilled in the art will recognize that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claims. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes. Any discussion of documents, acts, materials, devices, articles, and the like are included in the specification solely for the purpose of providing a context for the present invention. It is not suggested or represented that any or all of these matters form part of the prior art base or were common general knowledge in the field relevant to the present invention.

In this disclosure, whenever a composition or an element or a group of elements is preceded with the transitional phrase “comprising”, it is understood that we also contemplate the same composition, element or group of elements with transitional phrases “consisting of”, “consisting”, “selected from the group of” consisting of, “including”, or “is” preceding the recitation of the composition, element or group of elements and vice versa.

The present invention is described hereinafter by various embodiments with reference to the accompanying drawings, wherein reference numerals used in the accompanying drawing correspond to the like elements throughout the description. This invention may, however, be embodied in many different forms and should not be construed as limited to the embodiment set forth herein. Rather, the embodiment is provided so that this disclosure will be thorough and complete and will fully convey the scope of the invention to those skilled in the art.

In a brief explanation, the present invention may be understood as a multifunctional mixing container system configured to enhance the user experience of consuming various types of beverages or mixes within a defined or controlled temperature range. The present invention is configured to combine precision in temperature control, compartmentalized storage, interactive user interface, and a robust cleanliness assurance mechanism.

At its core, the container system consists of a container featuring internal grooves, capped or covered by a sip-and-seal lid that houses a toroidal removable ring and a user interface module. This structure accommodates one or more inner screw tubes, each configured to store one or more contents separately. The one or more inner screw tubes are configured to support stirring mechanism. The stirring mechanism includes the up and down movement of the one or more inner screw tubes along the grooves of the container for mixing of the one or more contents. The temperature range inside the container is achieved through a phase change material strategically placed within the toroidal ring and the space between the inner tubes and the container. Additionally, the outer surface of the container is coated with a temperature-sensitive paint that offers a visual representation of its temperature. To guarantee the hygiene of the container, a disinfectant module, equipped with UV light and/or ultrasonic unit, is positioned near the sip-and-seal lid on the underside of the container, ensuring the interior remains sanitized. The user interface module includes a touch-sensitive display unit, located either on the container or the lid, having one or more touch sensitive areas or input unit for input. The processing module, powered by a battery module. The processing module connected with the user interface and the disinfectant module, interpreting user commands. It is configured to process the input, and subsequently controlling both the sanitization process and the display updates.

For better understanding of different aspects of the invention, the present invention will now be described in detail with reference to accompanying drawings.

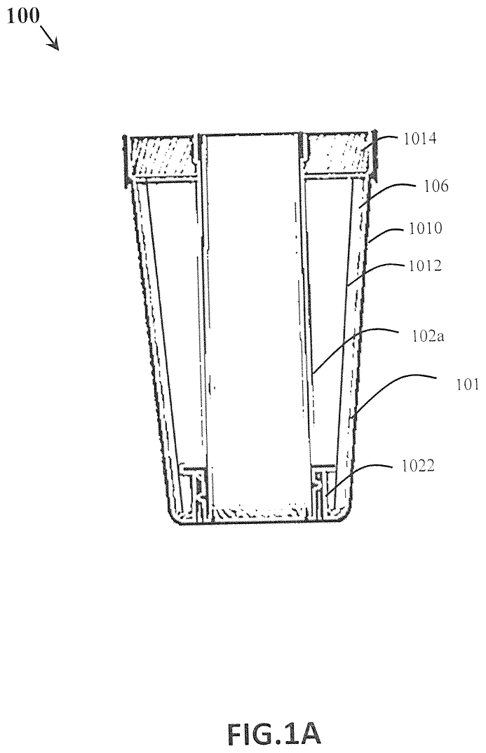

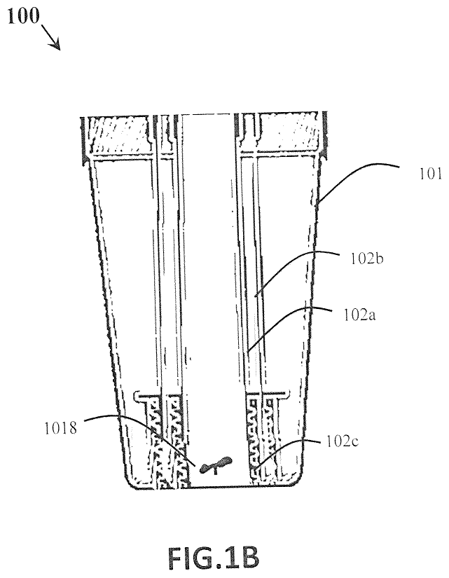

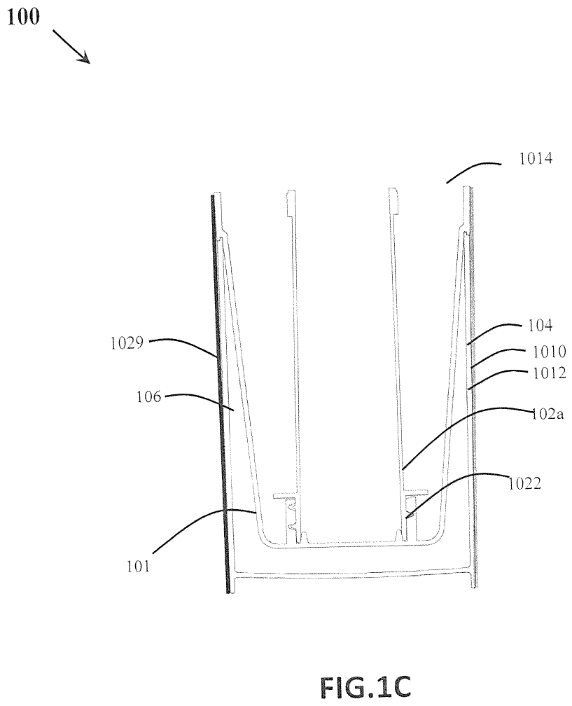

FIG. 1A illustrates a multi-compartment mixing container system for storing and mixing multiple contents at a predefined temperature range, in accordance with an embodiment of the present invention. As shown in FIG. 1A, the container system (100) comprises a container (101), one or more inner screw tubes (102a-102b) a battery (not shown) and a top cover (1014). The container (101) may include an outer surface (1010) and one or more inner surface (1012). The container (101) includes built-in one or more grooves (102c) inside the container (101). The one or more inner screw tubes (102a-102b) adapted to fit into the one or more grooves (102c) within the container (101).

The outer surface (1010) may be doubly insulated configured for heat insulation. The outer surface (1010) and the top cover (1014) may be selected from, but not limited to, double-walled stainless steel with vacuum insulation, high-quality BPA-free plastic with foam insulation, borosilicate glass with a silicone or neoprene sleeve, or combination thereof. It may further include a translucent section with a level indicator marking (not shown) for ingredient level monitoring. The one or more inner surface (1012) material selected from, but not limited to, PLA (Polylactic Acid), bamboo fibre, mushroom mycelium or even edible materials for single-use scenarios and hemp-based bioplastics. These materials are biodegradable, food safe and temperature resilience.

In some embodiments of the present invention, the outer surface (1010) of the container (101) may be coated with a temperature sensitive paint (1029). It is configured to indicate the temperature of the container system (100). The material selected for the temperature sensitive paint (1029) depends on parameters such as, but not limited to, the specific temperature range of interest, durability requirements, desired visual effects e.g., sharpness of colour change, range of colours, compatibility with the container material and/or cost constraints. The material may be selected from, but not limited to, liquid crystal thermochromic materials, leuco-dye-based thermochromic materials, thermochromic pigments, iron oxide thermochromic materials, vanadium dioxide (VO2), conductive polymers, polyaniline, liquid crystal thermochromic materials. It is configured to display the degree of hotness and coldness or temperature by varying between lighter and darker shades of colour to indicate temperature.

In an embodiment of the present invention, when the one or more inner screw tubes (102a-102b) are unscrewed or twisted or turned to activate the stirring mechanism, causing it to mix or blend the one or more contents thoroughly. As the one or more inner screw tubes (102a-102b) moved, it traverse up and down within the container, utilizing the one or more grooves (102c) as guiding paths. The motion effectively stirs and mixes the one or more contents stored within the one or more inner screw tubes (102a-102b). The one or more grooves (102c) are configured to ensure, not only the stability of the tubes but also guarantees consistent and even mixing throughout the container.

FIG. 1C illustrates an inner container of the multi-compartment mixing container system, in accordance with an embodiment of the present invention. In accordance with an additional or alternative embodiment of the present invention, the container (101) may be contained in an outer container (104). The container and the outer container may together form a cavity in between to fil in a suitable phase changing material (PCM) or ice.

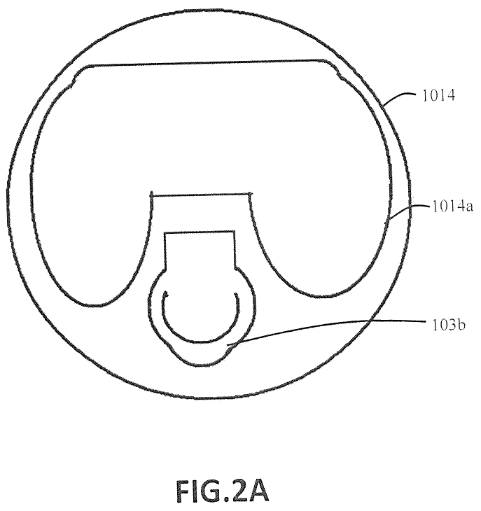

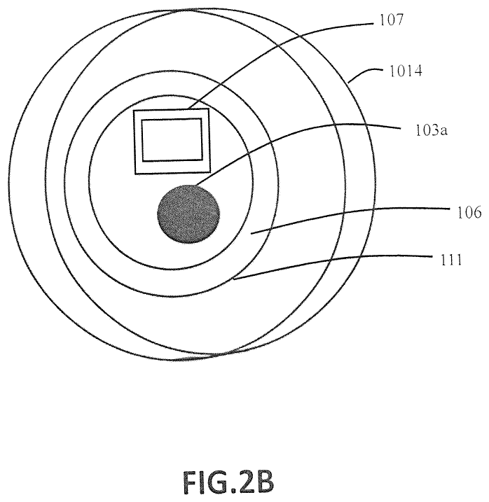

FIG. 2A illustrates top view of top cover (1014) of multi-compartment mixing container system (100), in accordance with an embodiment of the present invention. As shown in FIG. 2A, the lid or top cover (1014) on the top of the container (101) is configured to securely close the whole container (101), thereby ensuring the freshness and temperature stability of the one or more contents (not shown) inside. The top cover (1014) material can be selected from, but not limited to, high-quality BPA-free plastic, stainless steel, silicone or rubber, or borosilicate glass. The materials selected for constructing the top cover (1014) can vary depending on the desired features, durability, and insulation properties. The choice of material can also be influenced by the need for the lid to be dishwasher-safe, impact-resistant, or capable of providing an airtight seal. In some embodiment of the present invention, the top cover (1014) may also incorporate silicone or rubber gaskets to improve the sealing efficiency.

Additionally, In some embodiments of the present invention, the lid or top cover (1014) usually houses components, like a sip-and-seal lid (103b) for controlled liquid flow, a user interface module (1014a) for interaction with various functionalities of the container system (100), for example a disinfectant module (107) for sanitization, temperature sensitive paint (1029) to check temperature of liquid inside and turn on or off the stirring module (1018).

The sip-and-seal lid (103b) is configured to allow controlled and spill-resistant drinking. Unlike conventional lids, the sip-and-seal design incorporates a mechanism that allows the user to sip liquid directly from the container system (100) while minimizing the risk of spillage or leakage. This feature is particularly useful for on-the-go consumption of beverages, as it negates the need to remove the lid entirely, thereby reducing the chance of spills and maintaining the temperature and integrity of the liquid inside. When not in use, the opening is sealed to keep the liquid securely inside the container system (100). In some embodiments of the present invention the sip-and-seal lids (103b) may also incorporate additional features like straw hole as or ventilation ports (not shown) for smoother liquid flow.

The user interface module (1014a) is configured to elevate the user's experience by consolidating a host of interactive features. It includes one or more units selected from, but not limited to, a sound unit, a mic unit, and a touch sensitive display unit display equipped with a timer, clock, alarms, warnings, a stopwatch or combination thereof. The touchscreen or touch sensitive display unit not only simplifies navigation through these functionalities but also allows for greater convenience and customization. Users can easily control ingredient mixing, and disinfection processes with just an input such as a tap or a swipe from the one or more touch sensitive areas or input unit. The user interface module (1014a) may include from but not limited to capacitive, haptic screens, voice-activated and camera-based gesture operated, piezoelectric screens resistive touchscreens or combination thereof. The touch-enabled user interface or the user interface module (1014a) with the touch sensitive display unit enriches the user-friendliness and multifunctionality of the container system (100), catering to individual preferences and needs.

FIG. 2B illustrates bottom view of top cover (1014) of multi-compartment mixing container system (100), in accordance with an embodiment of the present invention. As shown in FIG. 2B, the top cover (1014) may include a disinfectant module (107) with a UV light unit and/or an ultrasonic unit, a hole (103a) at the back side of the sip-and-seal lid (103b) and a toroidal removable ring (111) at the bottom side. The disinfectant module (107) includes a UV light unit and/or an ultrasonic unit (not shown) configured to sanitize the interior surfaces of the container system (100) after and before its use. The UV light unit is configured to kill or inactivate microorganisms by destroying their nucleic acids and disrupting their DNA or RNA. It renders the microorganisms unable to perform vital cellular functions, thereby disinfecting the container system (100). The ultrasonic unit, on the other hand, uses high-frequency sound waves to create micro-bubbles in a liquid, which subsequently collapse. The process, known as cavitation, generates intense heat and pressure for a split second, sufficient to kill or disrupt various types of bacteria and microorganisms. The disinfectant module (107) is especially beneficial for users who want to ensure that their beverage or mix is being stored in a clean environment, free from any bacterial or viral contaminants.

The hole (103a) a drinking straw or a pipe (not shown) at the backside of the sip-and-seal lid (103b) is configured to provide a passageway for the liquid stored in the container system (100) to be easily consumed. It allows a user to sip beverages without removing the entire lid, thereby maintaining the temperature and other qualities of the drink. The hole (103a) is usually configured to fit most standard straw sizes and may also include a sealing mechanism or plug to close it off when not in use, ensuring that the container system (100) remains leak-proof and temperature-controlled. When the sip-and-seal lid (103b) is twisted or pressed, the force opens or aligns the hole (103a) with the pipe or straw that enables the user to get the mixture or the content inside the container system (100) out.

The hole (103a), configured to accommodate a drinking straw or a pipe (not shown), is located at the backside of the sip-and-seal lid (103b). It allows a convenient passageway for the user to sip liquids from the container system (100) without removing the entire lid. It maintains the temperature and other qualities of the drink intact. This hole (103a) is configured to fit a variety of standard straw sizes and may include a sealing feature or plug to close it when not in use. This ensures that the container system (100) remains leak-proof and maintains a controlled temperature. By twisting or pressing the sip-and-seal lid (103b), the hole (103a) aligns with the straw or pipe, allowing the user to easily access the one or more contents of the container system (100).

A phase change material, or PCM (106), may be incorporated within the one or more inner surfaces (1012) of the container system (100) and within the toroidal removable ring (111). The PCM is a specialized substance capable of changing its physical state, from solid to liquid or vice versa, at specific temperatures. Once the phase change occurs, the material remains in that state until further heat is either added or removed. This unique property makes PCMs highly applicable in various fields, particularly for thermal energy storage and management.

Further, as shown in FIG. 1C, in accordance with an embodiment of the present invention, in the cavity formed between the outer container (104) and the container (101), ice may be put to cool the liquid/beverages in the container (101). The temperature of the beverage may be maintained cool inside the container (101) using the ice contained in the cavity through conduction of the temperature. Further, the cavity may be filled with the PCM to keep the beverage inside the container (101) hot or cool.

The PCM (106) can be chosen from a range of materials including, but not limited to, paraffin wax, fatty acids, salt hydrates, water-based solutions, or other organic compounds. The PCM material used in the one or more inner surface (1012) and in the toroidal removable ring (111) may be the same or different, depending on its intended application. It can be used for either heating or cooling the contents of the container system (100), but generally not both simultaneously As the heat is absorbed, the PCM undergoes a phase change, transitioning from a solid to a liquid state. This phase change occurs at a specific temperature, which is the melting point of the PCM (106).

During this phase change, the PCM (106) effectively maintains a relatively constant temperature close to its melting point. For example, if the PCM (106) has a melting point of −5° C., it keeps the beverage close to −5° C. until it has completely melted.

For example, if we use sodium sulphate decahydrate as PCM for 1 litre of water, for getting liquid at temperature 5° C. from room temperature say 30° C., then: For the calculation of ΔT:
As ΔT=Final Temperature−Initial Temperature
(if say the specific heat capacity≈1.18 J/g° C., Melting point around 32° C.): Then, ΔT=5° C.−30° C.=−25° C.
And for the calculation of Q:

$$Q = m * ( - 25 ° C . ) * 1.18 J / g ° C .$$

$$Q = - 2 9.5 m J ( negative because heat is being absorbed )$$

Hence for the calculation of the mass (m) of Sodium Sulphate Decahydrate needed as PCM in the application:

$$m = ( - 29.5 J ) / ( - 25 ° C . * 1.18 J / g ° C . )$$

$$m \approx 100 g ( grams )$$

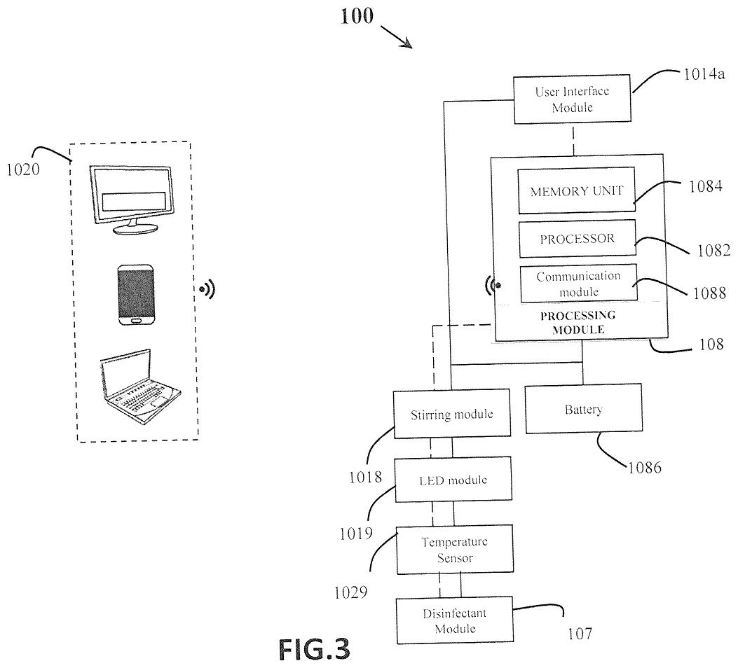

FIG. 3 illustrates functional of multi-compartment mixing container system (100), in accordance with an embodiment of the present invention. As shown in FIG. 3, the container system (100) can receive inputs from a user through the user interface module (1014a).

The one or more user devices (1020) may be selected from, but not limited to, a smartphone, tablet, Personal Desktop Assistant or PDA, laptop, desktop PC etc. In that sense, the plurality of user devices (102) is envisaged to include at least microprocessor, a connection means (ports for wired connections and Wi-Fi/Bluetooth modules for wireless connections) and input/output means such as display screen, keyboard/keypad or a touch input-based display.

In accordance with an embodiment of the present invention, the communication module (1088) is used in the container system (100) to connect the plurality of user devices (1020) and the processing module (108). The communication module (1088) can be a short-range and/or a long-range type, wire or wireless type communication module (1088). The communication interface includes, but not limited to, a serial communication interface, a parallel communication interface or a combination thereof. The communication module (1088) may be implemented using a number of protocols, such as but not limited to, TCP/IP, 3GPP, 3GPP2, LTE, IEEE 802.x etc. Preferably, the communication module (1088) is WIFI, Bluetooth or internet.

Further, the processing module (108) is configured to act as the central control unit or “brain” of the multi-compartment mixing container system (100). It is equipped with various components including a memory unit, a processor, and a communication module (1088). In various embodiments, the processor can be one of several types, but not limited to, a microprocessor, a general-purpose processor, an application-specific integrated circuit (ASIC), or a field-programmable gate array or FPGA. The memory unit may be selected from a group that comprises EPROM, EEPROM, and Flash memory for data storage. The processing module (108) is designed to interpret and execute user instructions received via the user interface module (1014a), coordinating these instructions with other container system (100) components like the stirring module (1018), battery, and temperature sensitive paint (1029). For example, if a user sets a specific temperature, the processing module (108) is configured to interface with the temperature sensitive paint (1029) to adjust the phase change material (106) to the desired setting. It can also activate the stirring module (1018) upon a user's request for mixing the contents. Furthermore, the processing module (108) manages battery usage for optimal performance, ensuring a smart, responsive, and user-friendly experience.

In accordance with an embodiment of the present invention, the processing module (108) may further include one or more modules selected from, but not limited to, a data analysis module, an incentive module and a notification module. In general, the word “module,” as used herein, refers to logic embodied in hardware or firmware, or to a collection of software instructions, written in a programming language, such as, for example, JavaScript, Python R, C, C#, Java, or Assembly. The modules described herein may be implemented as either software and/or hardware modules and may be stored in any type of computer-readable medium or other computer storage device.

In some embodiments of the present invention, the container system (100) may include a temperature sensitive paint (1029). The temperature sensitive paint (1029) is configured to continuously monitor the internal temperature of the one or more inner screw tubes (102a-102b) within the multi-compartment mixing container system (100). It sends real-time temperature data to the processing module (108), which then takes appropriate action to maintain the contents within a predefined temperature range. The predefined temperature range may be selected according to the user preference and content requirement for example it can be −2 to 40 degrees Celsius. The temperature sensitive paint is also configured to interface with the user interface module (1014a), providing temperature readouts that can be displayed for user awareness and control. It plays a crucial role in activating the phase change material (106) to either absorb or release heat, ensuring that the contents of the container system (100) are maintained at their optimal temperature for consumption.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope consistent with the principles and the novel and inventive features disclosed or suggested herein. Accordingly, the application is anticipated to hold on to all other such alternatives, modifications, and variations that fall within the scope of the present application and the appended claims.

1. A multi-compartment mixing container, the container system (100) comprising:
container (101) with built-in one or more grooves (102c) inside;
a sip-and-seal lid (103b) on the top of the container (101);
a toroidal removable ring (111) situated within the sip-and-seal lid (103b);
one or more inner screw tubes (102a-102b) adapted to fit into the one or more grooves (102c) within the container (101);
wherein the each of the inner removable screw tubes configured to separately store one or more contents;
a phase change material (106) disposed within the toroidal removable ring (111) and in the space between the inner removable screw tubes and the container (101);
a temperature sensitive paint (1029) on the surface of the container (101) to indicate the temperature of the container system (100);
a disinfectant module (107) with a UV light unit and/or an ultrasonic unit, placed proximal to the sip-and-seal lid (103b) on the bottom side of the container (101), to disinfect the inner surfaces of the container system (100);
a user interface module (1014a) disposed the container (101) or the sip-and-seal lid (103b), includes a touch sensitive display unit; wherein the touch sensitive display unit with one or more touch sensitive areas or input unit.
a stirring mechanism to facilitate mixing of the one or more contents stored in the one or more inner screw tubes (102a-102b) by twisting or turning along the one or more grooves (102c) up and down in the container system (100);
a processing module (108) connected to a battery module (108) 6; wherein the processing module (108) operatively connected to the user interface module (1014a), and the disinfectant module (107);
wherein the processing module (108) is configured to receive input from the user interface module (1014a);
process the input; and
switch the disinfectant module (107) and the user interface module (1014a).

2. The container system (100) as claimed in claim 1, wherein the touch sensitive display unit is configured to show a timer, a clock, warnings, clock time, stopwatch, a timer configured to notify users of optimal mixing times and displays nutritional values for combined ingredients.

3. The container system (100) as claimed in claim 1, wherein the user interface module (1014a) includes a sound unit, includes one or more input buttons, and, a communication module (1088).

4. The container system (100) as claimed in claim 1, wherein the one or more input buttons are selected from touch sensitive buttons, proximity switches, SPDT, DPDT or push buttons.

5. The container system (100) as claimed in claim 1, wherein the container (101) is doubly insulated and includes a translucent section and level indicator markings for ingredient level monitoring.

6. The container system (100) as claimed in claim 1, wherein the temperature sensitive paint (1029) on the surface of the container (101) is configured to display the degree of hotness and coldness or temperature by varying between lighter and darker shades of colour to indicate temperature.

7. The container system (100) as claimed in claim 1, wherein the material of the one or more inner screw tubes (102a-102b) is selected from eco-friendly materials, such as bamboo fibres, to promote sustainability.

8. The container system (100) as claimed in claim 1, wherein the container (101) and the one or more inner screw tubes (102a-102b) are foldable for convenient storage and transport.

9. The container system (100) as claimed in claim 1, wherein the phase change material (106) is selected from salt hydrate mixtures, eutectic mixtures, paraffin wax, potassium nitrate, fatty acids, organic based compounds or combination thereof.
